(19) United States
(12) Patent Application Publication
(10) Pub. No.: US 2026/0130362 A1
(43) Pub. Date: May 14, 2026

(54) Systems and Methods for Preservation of Biological Matter

(71) Applicants: Bridger Chytka, Thedford, NE (US); Tess Chytka, Thedford, NE (US); Brandi Chytka, Oshkosh, NE (US)

(72) Inventors: Bridger Chytka, Thedford, NE (US); Tess Chytka, Thedford, NE (US); Brandi Chytka, Oshkosh, NE (US)

(21) Appl. No.: 19/361,572

(22) Filed: Oct. 17, 2025

Related U.S. Application Data
(60) Provisional application No. 63/720,091, filed on Nov. 13, 2024.

(51) Int. Cl. A01N 1/162; A01N 1/145; A01N 1/165; C12N 5/00

(52) U.S. Cl. CPC A01N 1/162; A01N 1/145; A01N 1/165; C12N 5/545; C12N 5/562; C12N 5/565

(57) ABSTRACT

A system and method for preserving tissues, organs, and fluids by controlling pressure and temperature to extend viability. It utilizes a pressure vessel to prevent ice crystal formation, thereby avoiding cell wall rupture and death. Cellular respiration is suspended, enhancing storage time. The design includes both mobile and stationary units, allowing for flexible application. The mobile unit operates with a 12-volt battery, while the stationary unit can hold multiple specimens. This innovation aims to maintain the viability of biological materials for medical use, significantly increasing their storage duration and utility.

### RELATED APPLICATION

This application claims benefit to provisional application U.S. 63/720,091 filed on Nov. 11, 2024 and is incorporated in its entirety.

### BACKGROUND OF THE INVENTION

The present invention relates to systems and methods for the preservation of biological matter, specifically focusing on prolonging the useful life of mammalian tissues, organs, and fluids such as whole blood and blood components. Current standards for the storage of blood and its components involve mixing the blood with an anticoagulant and storing it in bags, which are then placed in coolers with ice packs to slow cellular respiration. However, this method has limitations, as cellular respiration continues, depleting cell energy, and frozen samples risk damage from oxidation and ice crystal formation, which can rupture cell walls.

### SUMMARY OF THE INVENTION

The present subject matter involves placing biological samples in a pressure vessel where they are subjected to high pressure, which allows the samples to be cooled to temperatures below freezing without forming ice crystals that could rupture cell walls. This method effectively suspends or greatly reduces cellular respiration, preserving the viability of cells. The system uses a hydraulic pump to apply pressure, and the samples are stored in a vacuum-sealed container to ensure consistent pressure application.

### BRIEF DESCRIPTION OF DRAWINGS

FIG. 1 shows a phase diagram of water.

FIG. 2 shows a top view of a first gen pressure vessel.

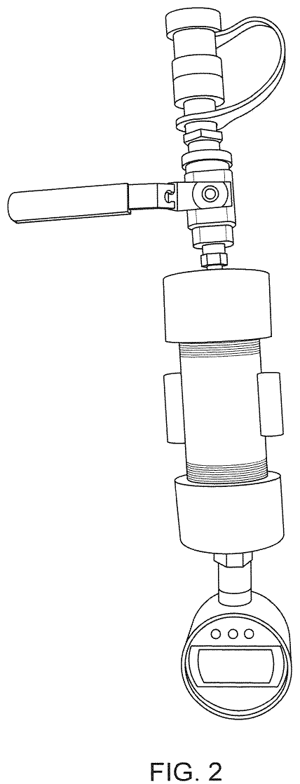

FIG. 3 shows a perspective view of a second gen pressure vessel.

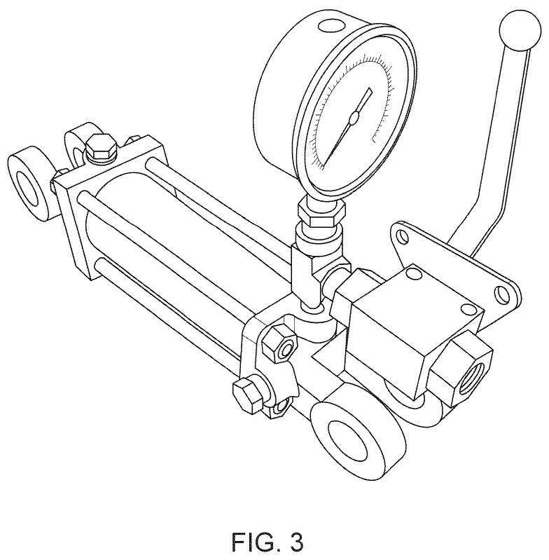

FIG. 4 shows a side view of a hydraulic pump.

FIG. 5 shows a perspective view of a freezer.

FIG. 6 shows a perspective view of a freezer.

FIG. 7 shows a top view of a programmable thermostat.

FIG. 8 shows a top view of a programmable thermostat.

FIG. 9 shows a schematic layout of a pressure vessel.

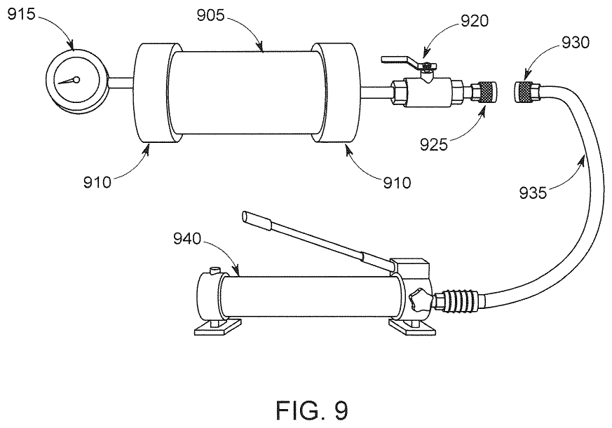

FIG. 10 shows a perspective view of a pressure vessel.

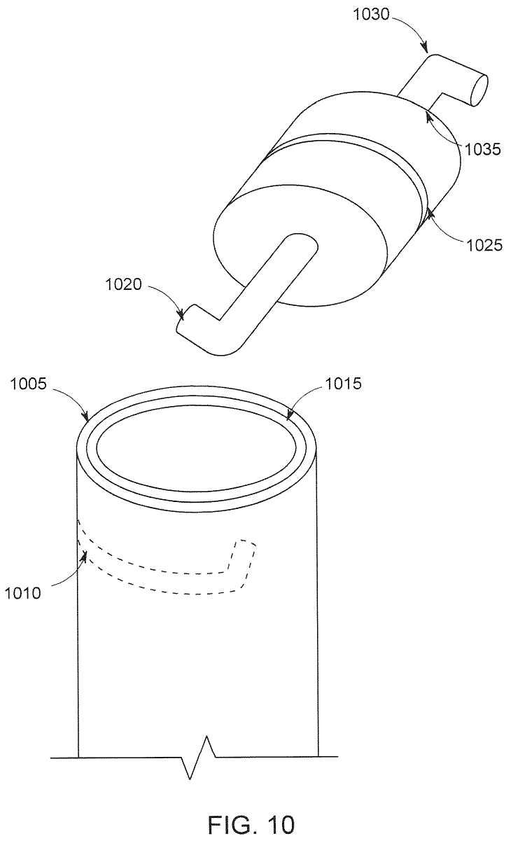

FIG. 11 shows a perspective view of a pressure vessel.

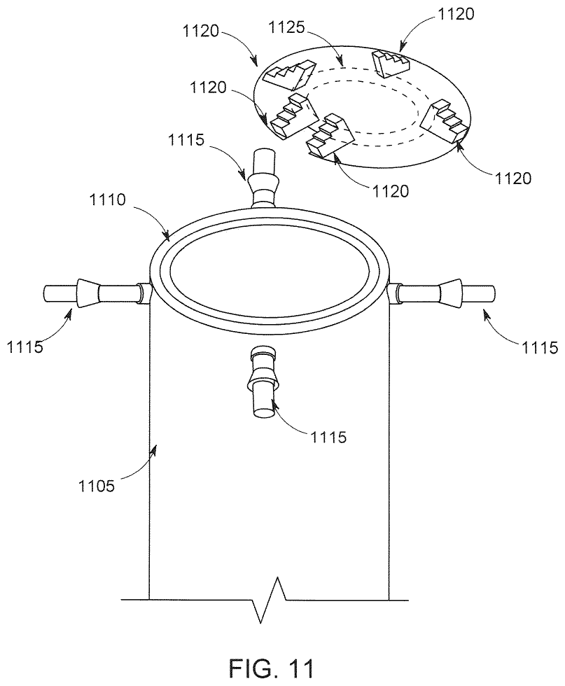

FIG. 12 shows a top view of a pressurized chamber container.

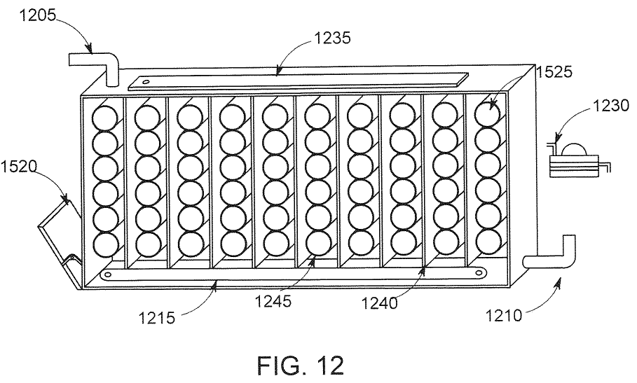

FIG. 13 shows a schematic view of a pressure chamber.

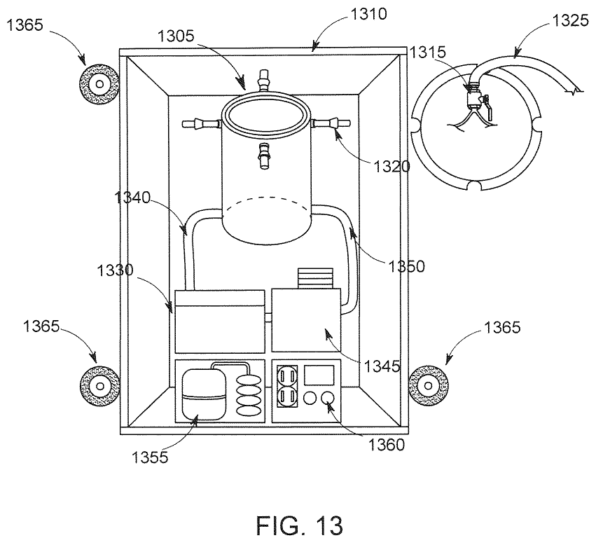

FIG. 14 shows a schematic view of a sample container.

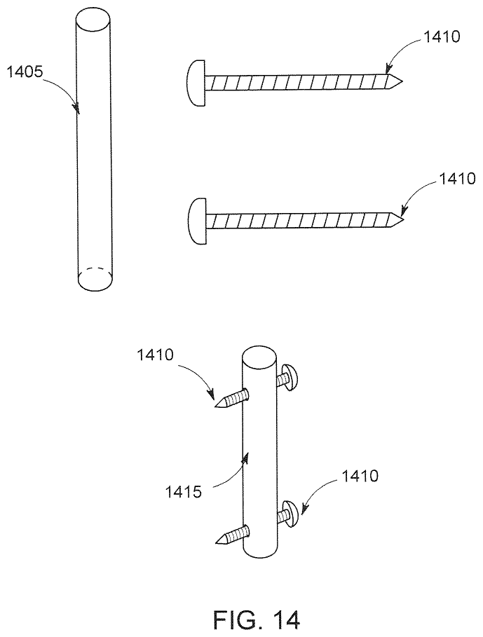

FIG. 15 shows a diagram of the correlation between temperature and pressure of water.

### DETAILED DESCRIPTION OF THE INVENTION

In the following description, numerous specific details are set forth to clearly describe various specific embodiments disclosed herein. One skilled in the art, however, will understand that the presently claimed invention may be practiced without all of the specific details discussed below. In other instances, well known features have not been described so as not to obscure the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings.

The phases of water are well documented. It is also well studied and proven that colder temperatures create hypothermia in cells which slows respiration. The novelty of the invention is putting blood, tissue, or organ under the correct pressure within the appropriate temperature.

There currently is no machine that stores pressurized cells that can later be “thawed” and retain their viability. The individual components of the machine are not new: an AC compressor, inverter, fluid pump, reservoir, insulated mobile container, and a stationary storage machine, are all devices that currently exist. But assembling them together for this purpose, in this configuration, is new.

The standard for storing whole blood is to place it in a bag with anticoagulant, then store said bag at room temperature, on a shelf. The problem with this type of storage is that the cells continue to use glucose and decay because the temperature of the blood encourages the cells to continue their vital life processes.

This loss of energy, and eventual death of the cell, leads to a short storage time and less viable productive blood or organ at the end of storage. The longer the blood or organ sits unused, the greater potential it has for causing adverse reactions to the recipient.

The short storage interval also means that hospitals are in a perpetual shortage of viable blood to administer to patients.

Organ storage and transportation for transplants is estimated within hours in most cases. The current standard is to flush the organ while still in the body, cool it off, and place it in a bucket or cooler containing ice water.

Hearts and lungs are viable outside of the body using current methods for only a few hours. Other organs have longer viable time periods, but are only a few days in length. The cause of loss of viability is due to cells in the tissues of the organ using up the energy that is present in the cell and blood that is present in the organ.

Another problem is death of cells and tissue that may come into contact with too cold of an environment when it is placed in the ice water bath. The cooling is not uniform and can cause cell death and ultimately the loss of viability if some areas are too cold (below 32 F the cell freezes and ruptures, or some areas too warm and they use up the cell's energy and eventually die).

By compressing the tissue or organ, then subsequently lowering the temperature to below freezing, we can slow down the use of energy by the cell, giving the organ or tissue a longer usable life.

With the compression and cooling method, you can also guarantee that the temperature of the environment is uniform and precise. This is beneficial because donor organs could potentially be transported farther from the donor location to the recipient location. Organs could even be stored to be used at a later date.

The following are some advantages of the present subject matter:

Increasing storage time for blood and blood components, increasing useful life of donated transplant organs.

Provide a stable environment to house blood, tissues, and organs.

The invention can be implemented throughout various elements of the system, including hardware elements, controller elements, and/or software elements. Although this description will often refer to certain embodiments, it will be understood that the concepts described herein can be applied in alternate embodiments and/or in different fields.

It is standard protocol of hospitals to store whole blood and blood components in bags. The blood is mixed with an anticoagulant by gently rocking the blood back and forth during collection.

Once the collection is complete, the blood is placed in a generic cooler, over ice packs, to keep it cool. The purpose of the ice pack is to slow the rate of cellular respiration and decrease the amount of energy used by red and white blood cells. After processing and breaking down of the blood into its different components, (whole blood, red blood cells, white blood cells, and plasma) the components are stored depending upon their component type.

Whole blood can also be stored at room temperature for 21-35 days in sealed plastic bags. Red blood cells can be refrigerated and stored up to 42 days. Platelets are stored for 5 days at room temperature with constant agitation to prevent clumping.

However, for samples stored at room temperature, cellular respiration is still taking place and depleting cell energy. For samples that are frozen, damage via oxidation and laceration may occur, especially to whole blood during transportation of cells, if they are in contact with freezing temperatures for too long.

When a mammalian cell is exposed to freezing temperature for too long, water present in the cell crystallizes and destroys the cell by rupturing the cell wall.

Donations of Kidney, Liver, and Pancreas bound to be placed in another living human are removed from the donor, flushed with the preservation solution and stored at 0-5 degrees Celsius for up to 2 days.

Heart transplants are limited to just 4-6 hours. During both time periods, the organs are placed in sealed plastic bags, then put into a cooler containing ice water. The purpose of the ice water is to slow the rate of cellular respiration.

A major risk with this type of storage and transportation, is the risk of ischemia, caused by the continued use of energy and loss of oxygenated blood to the tissues of the organ. Cells of the organ that come into direct contact with temperatures below 32 degrees Fahrenheit, will be destroyed by the formation of ice crystals and subsequent rupturing of the cell wall.

Water under normal atmospheric pressure, is known to have three phases: Vapor/Boiling—212 degrees Fahrenheit, liquid 32.1 to 211.9 degrees, and ice, 32 degrees Fahrenheit and lower.

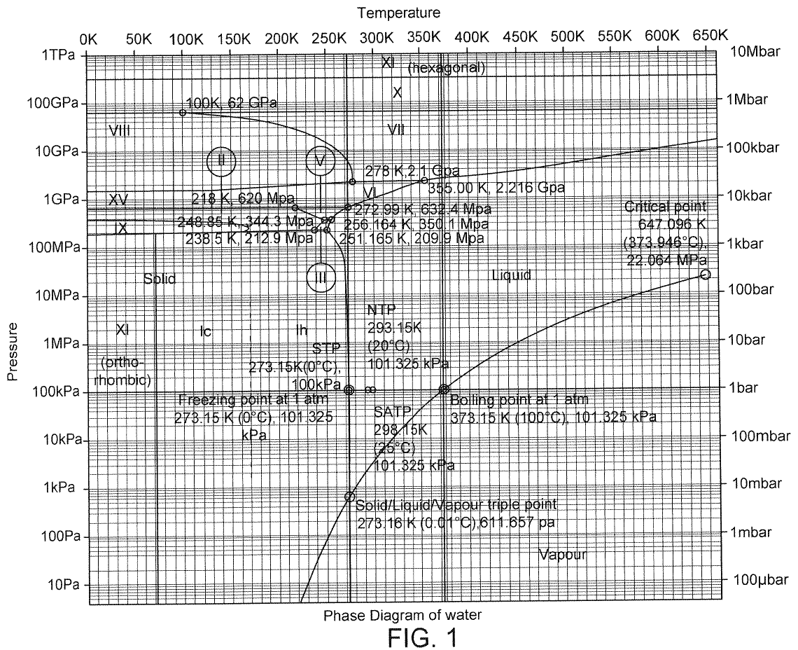

However, under extraordinary conditions, water's phases can be manipulated. FIG. 1 shows a diagram detailing the phases of water.

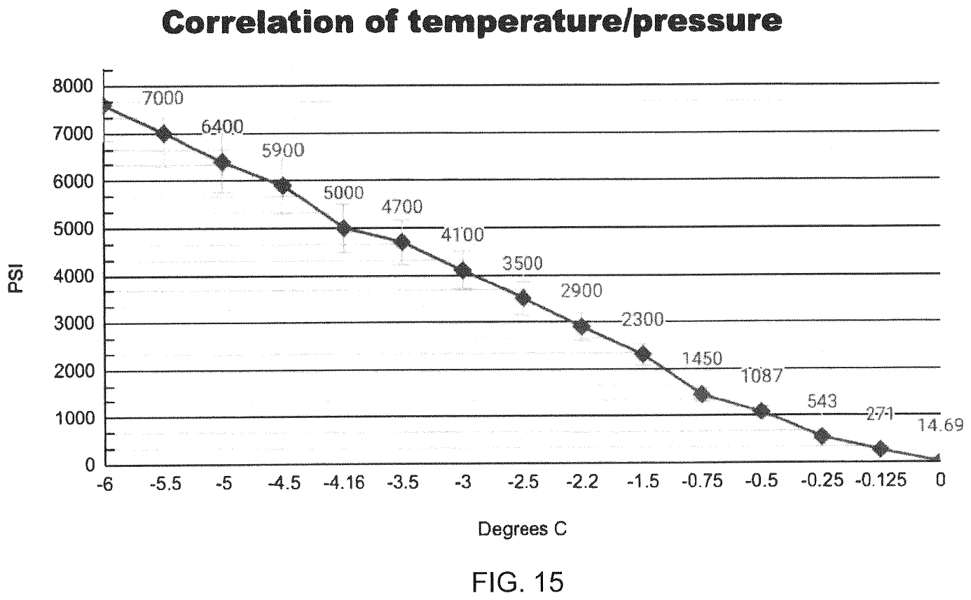

The present subject matter deals with the margin between liquid and ice at relatively high pressures. Water under 5 MPA 725 psi will remain liquid at 31.3 degrees Fahrenheit, 7.5 MPA 1,087.5 psi the temperature can be reduced to 30.9 degrees Fahrenheit, at 10 MPA 1,450 psi the temperature can be reduced to 30.6 degrees Fahrenheit, and as seen in FIG. 15, water under 7000 psi will remain liquid at 22.1 Fahrenheit or −5.5 degrees Celsius.

The forces exerted on the cell at the pressure and temperature ranges shown in FIG. 15 are not great enough to destroy the cell or change it morphologically.

It is well documented that mammalian cells exposed to hypothermia slow the rate of respiration and the conversion of glucose to ATP. The present subject matter shows a reduction in the use of glucose during pressurized sub-freezing temperatures. Removal from that environment yielded a return to previous glucose use levels.

The present subject matter demonstrates that the cells that were pressurized then “frozen” had their respiration suspended/greatly reduced then were able to be brought back to functioning levels.

By lowering the temperature below freezing, we are able to slow cellular respiration even further than with current techniques. This is because the addition of increased pressure on the cell keeps the water in the cell, in a liquid state, removing the possibility of ice crystal formation, cell wall rupture, and cell death.

The present subject matter demonstrates this concept physiologically by measuring blood glucose levels in control groups as well as pressurized groups.

Samples that were not pressurized, consequently froze and blood glucose levels remained unchanged. Whereas samples that were pressurized, then froze, had their rate of cellular respiration reduced.

In one embodiment, the pressure vessel comprises of a black schedule 80 2″×5″ pipe 905 threaded on both ends with NPT threads rated to 3000 psi, two mild steel caps 910 threaded with 2″ NPT threads rated to 4250 psi, 1500 psi pressure gauge 915, pipe nipple, ball valve 920 for closing/sealing chamber, female ⅜″ hydraulic quick connect coupling 925, a male ⅜″ hydraulic quick connect coupling 930, a ⅜″ 10,000 psi hydraulic hose 935, and a hand powered 10,000 psi hydraulic pump 940.

In another embodiment, a 6″×2″ “ag cylinder” the cylinder has been removed leaving only the outside canister, 2 O-ring seals at each end capped with a solid steel plate, which are held together with four bolts torqued to 30 PSI. A ball valve allows pressure in and seals the chamber once the desired pressure is reached.

In another embodiment, the present subject matter comprises of a pressure vessel 1005, machined descending groove 1010 which locks the lid into place, a machine lip with O-ring 1015, peg 1020 which fits a machined notch to lock the lid down in place, a machine lip 1025 to fit against the pressure vessel lip and O-ring to form a seal, a “T-handle” 1030 which may be pushed down and twisted to remove peg 1020, and a spring loaded “T-handle” 1035.

In another embodiment, the present subject matter comprises of a pressure vessel 1105, an O-ring 1110, flip up locking handles 1115 which fit between the ascending lock wherein the farther the “T” is pushed up the ascending lock, the tighter the lock; an ascending lock 1120 wherein the lock tightens with each step, and a machined groove 1125 which match the O-ring inside the pressure vessel.

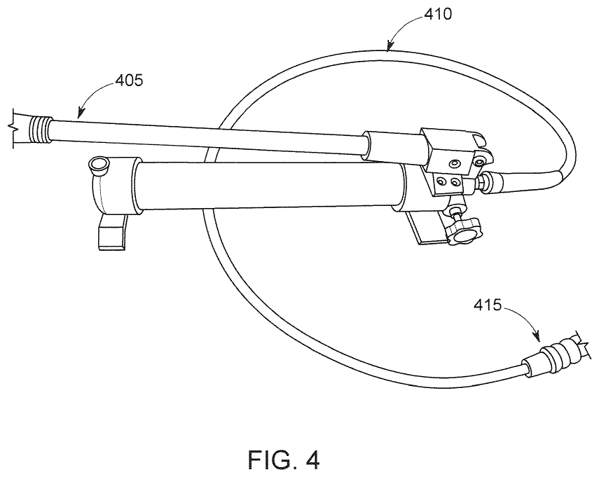

The present subject matter also comprises of a 10,000 psi hand pump 405, 10,000 psi hose 410, with a ⅜″ male hydraulic quick connect 415, as seen in FIG. 4.

The present matter also comprises of a hydraulic fluid further comprising of a 50-50 water and 99% isopropyl alcohol. The isopropyl alcohol insures no germs grow inside of the pressure vessel and in the event of failure with pressure, the canister will not burst due to formation of ice.

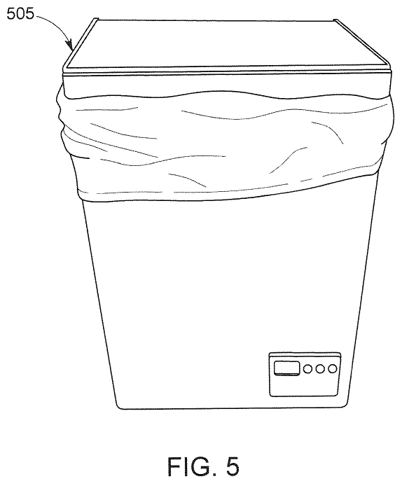

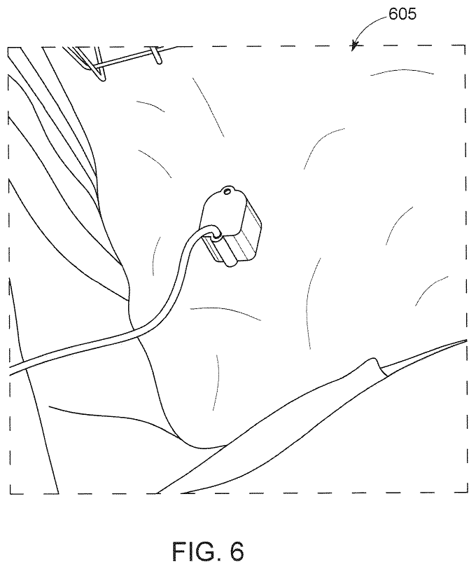

The present subject matter also comprises of deep freezer 505 filled with 50-50 mixture of water and isopropyl alcohol. The freezer 605 being filled with water/alcohol acts as a “thermal sink” to keep the compressor of the freezer from short cycling. FIG. 5 and FIG. 6 illustrate the type of freezer used.

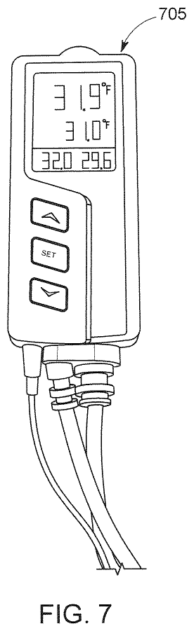

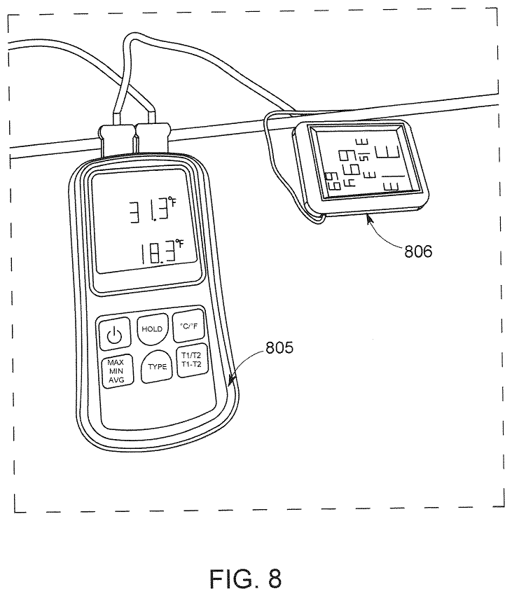

The present subject matter also comprises of a plurality of programmable thermostats 705 to keep liquid inside of the deep freeze at a temperature consistent with FIG. 15. FIG. 7 and FIG. 8 illustrate the redundant thermostats (805, 806) to ensure precise temperature control.

The present subject matter also comprises of a biological sample 1415, a vacuum sealed container wherein a ¼″ clear vinyl tubing 1405 is folded over on itself and held closed with a clamp, zip tie 1410, or other suitable mechanical closure at each end.

The container is pliable enough to have all of the air forced out of it. This is required because air will compress/move around and cause problems with maintaining a consistent pressure.

Hypothermia slows the rate of respiration of mammalian cells, but extreme cold is dangerous due to ice crystal formation inside of the cell, thus rupturing the cell wall, pressure is applied to the sample using a hydraulic pump to apply pressure consistent with FIG. 15.

In the present subject matter, this allows the sample to be cooled to approximately 10 degrees Fahrenheit below freezing without suffering damage from cell wall rupture or rupturing the cell from excessive pressure applied by the fluid.

Pascal's Law demonstrates that pressure applied to a fluid in a contained space is exerted equally and undiminished in all directions. The present subject matter uses a hydraulic pump to place organic samples under pressure.

The present subject matter uses a hydraulic fluid comprising of 50-50 water and isopropyl alcohol. Samples are placed in a vacuum sealed container with no air present within said container.

The presence of air in the container would skew the pressure on the sample because air compresses at a different rate than liquid.

Once the samples are in the pressure chamber, the chamber is pressurized, the inlet valve is closed, the sample is sealed and placed into a freezer with a steady temperature as low as 22.1 degrees Fahrenheit.

When taking the sample out of pressurization, the pressure chamber needs to be allowed to warm to above freezing, then the pressure is released from the chamber, and the sample can be removed.

The present subject matter's devices are made up of black steel schedule 80, 2 inch by 5 inch pipe with NPT pipe fitting caps on both ends or ag cylinder with O-rings and caps.

In one cap a pressure gauge is tapped and threaded, this doubles as a bleeder valve to ensure there is no air in the chamber and a port to screw in the pressure gauge.

On the other end in the center of the other cap is an inlet valve attached to the hydraulic pump to allow the chamber to be pressurized. The caps are stainless steel rated to 7000 psi.

The following is a list of steps to operate the present subject matter:

Sample is collected.

Sample is mixed with anticoagulant present inside the collection tube.

Sample is transferred to a vacuum container and sealed inside.

Samples are placed inside the pressure chamber.

Chamber is bled to ensure no air is present.

Chamber is filled with hydraulic fluid.

Chamber is attached to a hydraulic pump with a quick connect port.

Chamber is pressurized to be consistent with FIG. 15.

Inlet valve is closed.

Pressure is released from the pump (so fittings can be disconnected)

Pressure vessel is placed in the freezer at a temperature consistent with FIG. 15.

In some embodiments, the present subject matter is a self-contained mobile unit comprising of a pressure vessel with removable lid 1305, a portable insulated case 1310, a ball valve 1315 to let air out, a swivel bolt 1320 with wing nut to lock down the lid, an overflow hose 1325 which takes excess fluid and air to a reservoir, a reservoir 1330 for hydraulic fluid, a return line 1340, a hydraulic pump 1345, a pressurized line 1350 to the pressure vessel, a cooling unit 1355, a power supply 1360, and wheels 1365, wherein the unit operates from a 12-volt battery and inverter that powers the compressor and condenser to cool the fluid that is used to exert pressure on the blood or organ, as well as keep the pressure vessel at temperature, once it is pressurized.

The outside container is comprised from a heavy-duty plastic that is insulated between the inside and outside walls to keep the amount of time the compressor is cycling low.

In some embodiments, the mobile unit is integrated with a computer program to allow the user to put the sample in the pressure vessel, when the cap is sealed, and the device bleeds itself, pressurizes, and maintains temperature automatically.

In other embodiments, the present subject matter is a unit that is stationary, comprising of a 50-50 isopropyl/water inlet 1205, a cooled 50-50 isopropyl/water outlet 1210, a conveyor 1215, an exit 1220, a plurality of organic samples 1225, a fluid circulation pump 1230, a door 1235 to load samples, and a selection lever 1240 wherein the lever places the container 1245 onto the conveyer. The unit is filled with circulating fluid that is held at a temperature consistent with FIG. 15 to ensure samples stay at the correct temperature.

This embodiment is able to hold multiple specimens. Once the units are pressurized, they are placed in the stationary unit according to their size and type, which determined the size of pressure vessel needed, and therefore which column they would be placed in.

The samples are stacked vertically in columns above a conveyor belt. This embodiment is similar to a vending machine that allows you to make a selection and have the sample delivered to the exterior of the machine. A sample could be selected from a keypad on the outside of the machine, a lever opens at the bottom of the column which allows the sample to be lowered onto the conveyor belt and delivered to the exit. Once the sample is outside of the machine, it should be allowed to warm to room temperature before it is depressurized and opened.

In yet another embodiment, the vessels are built out of carbon fiber, aluminum, or some other lightweight material that is capable of withstanding the amount of required pressure.

Under current testing, the present subject matter has been tested up to 7000 psi and has been able to retain morphological and physiological resuscitation of the cell.

Schedule 80 pipe has a working pressure 8 times greater than its rated pressure. That means the working pressure of the new material needs to be 1,500 psi with a burst pressure of 12,000 psi.

In some embodiments, the lid and caps are snap fit or push lock lid with an O ring seal that can be applied and removed by hand.

While this specification contains many specific implementation details, these should not be construed as limitations on the scope of any inventions or of what may be claimed, but rather as descriptions of features specific to particular implementations of particular inventions.

Certain features that are described in this specification in the context of separate implementations can also be implemented in combination in a single implementation. Conversely, various features that are described in the context of a single implementation can also be implemented in multiple implementations separately or in any suitable sub combination.

Moreover, although features may be described above as acting in certain combinations and even initially claimed as such, one or more features from a claimed combination can in some cases be excised from the combination, and the claimed combination may be directed to a sub combination or variation of a sub combination.

Similarly, while operations may be depicted in the drawings in a particular order, this should not be understood as requiring that such operations be performed in the particular order shown or in sequential order, or that all illustrated operations be performed, to achieve desirable results.

In certain circumstances, multitasking and parallel processing may be advantageous. Moreover, the separation of various system components in the implementations described above should not be understood as requiring such separation in all implementations, and it should be understood that the described components and systems can generally be integrated together in a single product or packaged into multiple products.

Each numerical value presented herein is contemplated to represent a minimum value or a maximum value in a range for a corresponding parameter.

Accordingly, when added to the claims, the numerical value provides express support for claiming the range, which may lie above or below the numerical value, in accordance with the teachings herein.

Every value between the minimum value and the maximum value within each numerical range presented herein (including in the figures), is contemplated and expressly supported herein, subject to the number of significant digits expressed in each particular range. Absent express inclusion in the claims, each numerical value presented herein is not to be considered limiting in any regard.

Unless expressly described elsewhere in this application, as used herein, when the term “substantially” or “about” is before a quantitative value, the present disclosure also includes the specific quantitative value itself, as well as, in various cases, a ±1%, ±2%, ±5%, and/or ±10% variation from the nominal value unless otherwise indicated or inferred.

Having described herein illustrative embodiments, persons of ordinary skill in the art will appreciate various other features and advantages of the invention apart from those specifically described above. It should therefore be understood that the foregoing is only illustrative of the principles of the invention, and that various modifications and additions, as well as all combinations and permutations of the various elements and components recited herein, can be made by those skilled in the art without departing from the spirit and scope of the invention.

Accordingly, the appended claims shall not be limited by the particular features that have been shown and described, but shall be construed also to cover any obvious modifications and equivalents thereof.

1. A system for preserving mammalian tissues, organs, and fluids, comprising:
a pressure vessel configured to maintain a predetermined pressure sufficient to prevent ice crystal formation within the biological material;
a temperature control unit operatively connected to the pressure vessel, configured to maintain the biological material at a sub-freezing temperature without causing cellular damage;
a mobile unit housing the pressure vessel and temperature control unit, equipped with a power source to enable operation during transport;
wherein the system is designed to extend the viability of the biological material by reducing cellular respiration and preventing ice crystal formation.

2. The system of claim 1, wherein the pressure vessel is constructed from a material capable of withstanding pressures up to 7,000 psi.

3. The system of claim 1, wherein the temperature control unit maintains the biological material at a temperature between 22.1 degrees Fahrenheit and 32.0 degrees Fahrenheit.

4. The system of claim 1, further comprising a hydraulic pump to apply pressure to the pressure vessel.

5. The system of claim 1, wherein the mobile unit includes an insulated container to minimize temperature fluctuations during transport.

6. The system of claim 1, wherein the mobile unit is powered by a 12-volt battery and inverter.

7. The system of claim 1, further comprising a computer program integrated with the mobile unit to automate pressure and temperature control.

8. The system of claim 1, wherein the pressure vessel includes a quick-connect coupling for easy attachment to the hydraulic pump.

9. The system of claim 1, wherein the pressure vessel is equipped with a pressure gauge to monitor internal pressure.

10. A method for preserving tissues, organs, and fluids, comprising the steps of:
placing the biological material in a pressure vessel;
applying pressure to the biological material within the pressure vessel to maintain a pressure sufficient to prevent ice crystal formation;
controlling the temperature of the biological material to a sub-freezing level using a temperature control unit; and
maintaining the biological material in the pressure vessel under the controlled temperature and pressure conditions to extend its viability.

11. The method of claim 10, further comprising the step of sealing the biological material in a vacuum-sealed container before placing it in the pressure vessel.

12. The method of claim 10, wherein the pressure applied is consistent with FIG. 15.

13. The method of claim 10, further comprising the step of gradually releasing the pressure from the pressure vessel before removing the biological material.

14. The method of claim 10, wherein the biological material is selected from the group consisting of whole blood, red blood cells, white blood cells, plasma, and organs.

15. The method of claim 10, further comprising the step of using a programmable thermostat to maintain the desired temperature range.

16. A stationary system for preserving mammalian tissues, organs, and fluids, comprising:
a pressure vessel configured to maintain a predetermined pressure;
a temperature control unit configured to maintain the biological material at a sub-freezing temperature;
a stationary unit housing multiple pressure vessels, each operatively connected to the temperature control unit;
a conveyor mechanism for transporting the biological material to and from the pressure vessels;
wherein the system is designed to extend the viability of the biological material by reducing cellular respiration and preventing ice crystal formation.

17. The stationary system of claim 16, wherein the conveyor mechanism is automated to deliver the biological material to a designated location upon user selection.

18. The stationary system of claim 16, further comprising a user interface for selecting and retrieving specific biological materials.

19. The stationary system of claim 16, wherein the pressure vessels are arranged in columns based on the size and type of biological material.

20. The stationary system of claim 16, wherein the temperature control unit circulates a cooling fluid to maintain the desired temperature.
